SEQUENCE LISTING (4 sequences)

SEQ ID NO: 1 - protein, 4 aa, Artificial Sequence
  Synthetic
IVFK

SEQ ID NO: 2 - protein, 4 aa, Artificial Sequence
  Synthetic
  SITE (3)..(3): Xaa is cyclohexylalanine
IVXK

SEQ ID NO: 3 - protein, 4 aa, Artificial Sequence
  Synthetic
KFVI

SEQ ID NO: 4 - protein, 4 aa, Artificial Sequence
  Synthetic
  SITE (2)..(2): Xaa is cyclohexylalanine
KXVI

(19) United States
(12) Patent Application Publication
(10) Pub. No.: US 2024/0261465 A1
(43) Pub. Date: Aug. 8, 2024

(54) METHOD OF STOPPING BLEEDING USING NANOFIBROUS PEPTIDE HYDROGELS AS HEMOSTASIS AGENT

(71) Applicant: KING ABDULLAH UNIVERSITY OF SCIENCE AND TECHNOLOGY, Thuwal (SA)

(72) Inventors: Sherin ABDELRAHMAN, Thuwal (SA); Hamed ALBALAWI, Thuwal (SA); Salwa Ahmed ALSHERI, Thuwal (SA); Charlotte A.E. HAUSER, Thuwal (SA)

(21) Appl. No.: 18/565,644

(22) PCT Filed: Jun. 3, 2022
(86) PCT No.: PCT/IB2022/055194

§ 371 Date: Nov. 30, 2023

Related U.S. Application Data
(60) Provisional application No. 63/196,787, filed on Jun. 4, 2021.

(51) Int. Cl. A61L 26/00; B33Y 30/00

(52) U.S. Cl. CPC A61L 26/0047; A61L 26/008; B33Y 30/00; A61L 2300/25; A61L 2300/418; A61L 2400/04

(57) ABSTRACT

The present disclosure relates generally to a method of stopping bleeding comprising applying a peptide-based hemostatic material to a bleeding wound, wherein the peptide-based hemostatic material comprises at least one self-assembling ultrashort peptide, and wherein an application of the peptide-based hemostatic material to the bleeding wound produces a coagulation time of less than 40 seconds. The present disclosure also provides a hand-held device for the application of the peptide-based hemostatic material.

### CROSS-REFERENCE TO RELATED APPLICATIONS

This application claims benefit of priority of U.S. Provisional Patent Application No. 63/196,787 entitled, “Nanofibrous Peptide Hydrogels as Hemostasis Agent” filed Jun. 4, 2021. The entire contents and disclosures of these patent applications are incorporated herein by reference in their entirety.

This application makes reference to U.S. patent application Ser. No. 17/401,542, entitled “SCAFFOLDS FROM SELF-ASSEMBLING TETRAPEPTIDES SUPPORT 3D SPREADING, OSTEOGENIC DIFFERENTIATION AND ANGIOGENESIS OF MESENCHYMAL STEM CELLS,” filed Aug. 13, 2021. The entire contents and disclosures of these patent applications are incorporated herein by reference.

### REFERENCE TO A “SEQUENCE LISTING”

The present application includes a Sequence Listing which has been submitted electronically in an ASCII text format. This Sequence Listing is named 114147-24071WO01_sequence listing.TXT was created on Jan. 18, 2022, is 997 bytes in size and is hereby incorporated by reference in its entirety.

### BACKGROUND

### Field of the Invention

The present disclosure relates generally to a method of stopping bleeding within a few seconds.

### Background of the Invention

Hemorrhage is one of the greatest reasons for mortality as more than 85% of deaths from survivable wounds are because of severe, uncontrolled bleeding. Traumatic wounds may lead to coagulopathy, where clotting is weakened due to systemically decreased blood flow. This in turn causes anticoagulation and increased degradation of fibrin, the main factor of clot formation (Hsu, B. B., et al., Clotting mimicry from robust hemostatic bandages based on self-assembling peptides. ACS nano, 2015. 9(9): p. 9394-9406). Controlling blood loss during and after surgical procedures of parenchymal organs, for example the liver, kidney, and spleen, is difficult and is even fatal if the injury becomes severe (Song, H., L. Zhang, and X. Zhao, Hemostatic efficacy of biological self-assembling peptide nanofibers in a rat kidney model. Macromolecular bioscience, 2010. 10(1): p. 33-39).

As the prevalence of large traumatic injury rises, it is essential to create a hemostatic material that can assist with blood clotting through a mechanism independent of the body's own coagulation system so that hemostasis may be succeeded in spite of coagulopathy (Kheirabadi, B., Evaluation of topical hemostatic agents for combat wound treatment. US Army Medical Department Journal, 2011). However, the established hemostatic techniques such as pressure, string, and cautery have some limitations for such injuries and are often unpractical (Holcomb, J. B., et al., Implications of new dry fibrin sealant technology for trauma surgery. Surgical Clinics, 1997. 77(4): p. 943-952). Furthermore, several topical hemostatic products have been developed recently, including microcrystalline collagen, absorbable gelatin sponges, oxidized cellulose sponges, and fibrin sealants (Albala, D. M., Fibrin sealants in clinical practice. Cardiovascular surgery, 2003. 11: p. 5-11; Plaisier, B. R. Surgical perspectives to control bleeding in trauma. in Seminars in Anesthesia, Perioperative Medicine and Pain. 2001. Elsevier; Kheirabadi, B. S., et al., Comparative study of the efficacy of the common topical hemostatic agents with fibrin sealant in a rabbit aortic anastomosis model. Journal of Surgical Research, 2002. 106(1): p. 99-107; Cole, D. J., et al., A pilot study evaluating the efficacy of a fully acetylated poly-N-acetyl glucosamine membrane formulation as a topical hemostatic agent. Surgery, 1999. 126(3): p. 510-517; Tuthill, D. D., et al., Assessment of topical hemostats in a renal hemorrhage model in heparinized rats. Journal of Surgical Research, 2001. 95(2): p. 126-132). However, there are disadvantages for each of these hemostatic agents. For example, because collagen, fibrinogen, and thrombin are commonly sourced from animal or human blood, there is a substantial risk of viral infection, while preparation of fibrin hydrogels from fibrin sealant is expensive and time-consuming. Thus, these materials are not suitable for use in surgery. As a result, it is crucial to fabricate a new hemostatic agent that is safer, more efficient, cheaper, easy and fast to prepare (Song, H., L. Zhang, and X. Zhao, Hemostatic efficacy of biological self-assembling peptide nanofibers in a rat kidney model. Macromolecular bioscience, 2010. 10(1): p. 33-39).

An alternative approach to establishing hemostasis is through the use of peptide-based hydrogels. Hydrogels are a material class made up of nanofibrous networks with a high water retention capacity. Their physical properties such as swelling, permeation, mechanical, surface, and optical characteristics can be modulated (Crescenzi, V., et al., Synthesis and partial characterization of hydrogels obtained via glutaraldehyde crosslinking of acetylated chitosan and of hyaluronan derivatives. Biomacromolecules, 2003. 4(4): p. 1045-1054). These peptides contain a polar head group and a hydrophobic tail and fall into a unique class of amphiphilic ultrashort peptides with an innate propensity to self-assemble into three-dimensional nanofibrous hydrogels. The fiber structures bear a strong resemblance to the extracellular matrix (ECM) particularyof the collagen fibrlis. Because of their amphiphilic nature, these fibrous peptide networks entrap (exceeding 99%) water (Loo, Y., et al., Peptide bioink: self-assembling nanofibrous scaffolds for three-dimensional organotypic cultures. Nano letters, 2015. 15(10): p. 6919-6925). In solution, these peptides form nanofibers that can quickly enhance blood coagulation (Ellis-Behnke, R., At the nanoscale: nanohemostat, a new class of hemostatic agent. Wiley interdisciplinary reviews: nanomedicine and nanobiotechnology, 2011. 3(1): p. 70-78). For example, RADA16 is a commercially synthesized and purified peptide that is capable of self-assembly. RADA16 is an ionic self-complementary peptide, in which the positively charged arginine (R) and negatively charged aspartate (D) are arranged alternatively. RADA16 self-assembles through a sliding diffusion mechanism, based on the ionic interaction between the positively and negatively charged amino acids (Yokoi, H., T. Kinoshita, and S. Zhang, Dynamic reassembly of peptide RADA16 nanofiber scaffold. Proceedings of the National Academy of Sciences of the United States of America, 2005. 102(24): p. 8414-8419). However, the applications of RADA16 3-D matrix are very limited, because of high production costs. Moreover, due to its acidity, the pH of self-assembled RADA16 hydrogel needs to be adjested before used. Therefore, an improved peptide-based hemostasis material is needed.

### SUMMARY

According to a first broad aspect the present disclosure provides a method of stopping bleeding comprising: applying a peptide-based hemostatic material to a bleeding wound, wherein the peptide-based hemostatic material comprises at least one self-assembling ultrashort peptide, and wherein an application of the peptide-based hemostatic material to the bleeding wound produces a coagulation time of less than 40 seconds.

According to a second broad aspect the present disclosure provides a method of stopping bleeding comprising: applying a peptide-based hemostatic material to a bleeding wound, wherein the peptide-based hemostatic material comprises at least one self-assembling ultrashort peptide, wherein an application of the peptide-based hemostatic material to the bleeding wound produces a coagulation time of less than 40 seconds, wherein the self-assembling ultrashort peptide has a general formula selected from: AₙBₘX, BₘAₙX, XAₙBₘ and XBₘAₙ, wherein the total number of amino acids of the self-assembling ultrashort peptide does not exceed 7 amino acids; wherein A is an aliphatic amino acids, selected from the group consisting of: isoleucine, leucine or any combination thereof, with n being an integer being selected from 0-5; wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3; wherein X is comprised of a polar amino acid, selected from the group consisting of: aspartic acid, glutamic acid, lysine, arginine, histidine, cysteine, serine, threonine, asparagine, and glutamine.

Other aspects and features of the present disclosure will become apparent to those skilled in the art upon review of the following description of specific embodiments of the invention in conjunction with the accompanying figures.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The accompanying drawings, which are incorporated herein and constitute part of this specification, illustrate exemplary embodiments of the invention, and, together with the general description given above and the detailed description given below, serve to explain the features of the invention.

FIG. 1 is a graph showing the gelation time of IVZK (SEQ ID NO. 2) at different concentration according to an embodiment of the present disclosure.

FIG. 2 is a graph showing the gelation time of IVFK (SEQ ID NO. 1) at different concentration according to an embodiment of the present disclosure.

FIG. 3 is a graph showing onset of gelation of IVZK (SEQ ID NO. 2) at different temperatures according to an embodiment of the present disclosure.

FIG. 4 is a graph showing onset of gelation of IVFK (SEQ ID NO. 1) at different temperatures according to an embodiment of the present disclosure.

FIG. 5 is a graph showing the partial thromboplastin time (aPTT) test results demonstrating the dramatic decrease in coagulation time after the addition of peptide solution pointing to the important effect of those peptides in hemostasis for deficiency coagulation factors plasma according to an embodiment of the present disclosure.

FIG. 6 is a graph showing the partial thromboplastin time (aPTT) test results demonstrating the dramatic decrease in coagulation time after the addition of peptide solution pointing to the important effect of those peptides in hemostasis for normal plasma according to an embodiment of the present disclosure.

FIG. 7 is a photo showing the morphology of the nanofibers of the IVFK (SEQ ID NO. 1) hydrogels in water according to an embodiment of the present disclosure.

FIG. 8 is a photo showing the morphology of the nanofibers of the IVZK (SEQ ID NO. 2) hydrogels in water according to an exemplary embodiment of the present disclosure.

FIG. 9 is a photo showing the morphology of the nanofibers of the IVFK (SEQ ID NO. 1) hydrogels in PBS according to an exemplary embodiment of the present disclosure.

FIG. 10 is a photo showing the morphology of the nanofibers of the IVZK (SEQ ID NO. 2) hydrogels in PBS according to an exemplary embodiment of the present disclosure.

FIG. 11 is a photo showing the morphology of the nanofibers of the IVFK (SEQ ID NO. 1) hydrogels with plasma according to an exemplary embodiment of the present disclosure.

FIG. 12 is a photo showing the morphology of the nanofibers of the IVZK (SEQ ID NO. 2) hydrogels with plasma according to an exemplary embodiment of the present disclosure.

FIG. 13 is a photo showing the morphology of the fibers of the Fibrin according to an exemplary embodiment of the present disclosure.

FIG. 14 is a photo showing the IVFK (SEQ ID NO. 1) nanofiber morphology after mixing with blood samples according to an exemplary embodiment of the present disclosure.

FIG. 15 is a photo showing the IVZK (SEQ ID NO. 2) nanofiber morphology after mixing with blood samples according to an exemplary embodiment of the present disclosure.

FIG. 16 is an enlarged photo showing the IVFK (SEQ ID NO. 1) nanofiber morphology after mixing with blood samples according to an exemplary embodiment of the present disclosure.

FIG. 17 is an enlarged photo showing the IVZK (SEQ ID NO. 2) nanofiber morphology after mixing with blood samples according to an exemplary embodiment of the present disclosure.

FIG. 18 is a photo showing the morphology of Fibrin fibers after mixing with blood samples according to an exemplary embodiment of the present disclosure.

FIG. 19 is an enlarged photo showing the morphology of Fibrin fibers after mixing with blood samples according to an exemplary embodiment of the present disclosure.

FIG. 20 is a photo showing the absence of agglutination activity without the addition of peptide hydrogel according to an exemplary embodiment of the present disclosure.

FIG. 21 is a photo showing the agglutination activity of IVZK (SEQ ID NO. 2) according to an exemplary embodiment of the present disclosure.

FIG. 22 is a photo showing the agglutination activity of IVFK (SEQ ID NO. 1) according to an exemplary embodiment of the present disclosure.

FIG. 23 is fluorescence microscopy images showing platelet adhered on different coating surfaces according to an exemplary embodiment of the present disclosure.

FIG. 24 is a graph showing the fluorescence intensity of platelets adhered to different surfaces according to an exemplary embodiment of the present disclosure.

FIG. 25 is a photo showing the viability of human bone marrow-derived mesenchymal stem cells (BM-MSCs) assessed by the live/dead stain on day one, and seven according to an exemplary embodiment of the present disclosure.

FIG. 26 is a photo showing the application of IVFK (SEQ ID NO. 1) and IVZK (SEQ ID NO. 2) as hemostatic materials according to an exemplary embodiment of the present disclosure.

FIG. 27 is a figure showing the front view of the portable 3D printer according to an exemplary embodiment of the present disclosure.

FIG. 28 is a figure showing the bottom view of the portable 3D printer according to an exemplary embodiment of the present disclosure.

FIG. 29 is a figure showing the side view of the portable 3D printer according to an exemplary embodiment of the present disclosure.

FIG. 30 is a figure showing the back view of the portable 3D printer according to an exemplary embodiment of the present disclosure.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising.” the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing figures may be flipped over, rotated by 90° in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present disclosure, the term “amphiphilic” or “amphiphilicity” refers to being a compound consisting of molecules having a water-soluble group at one end and a water-insoluble group at the other end.

For purposes of the present disclosure, the term “aliphatic” means, unless otherwise stated, a straight or branched hydrocarbon chain, which may be saturated or mono- or poly-unsaturated and include heteroatoms. An unsaturated aliphatic group contains one or more double and/or triple bonds (alkenyl or alkynyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to 5, to 10, to 15 or to 20 carbon atoms. Examples of alkenyl radicals are straight-chain or branched hydrocarbon radicals which contain one or more double bonds. Alkenyl radicals generally contain about two to about twenty carbon atoms and one or more, for instance two, double bonds, such as about two to about ten carbon atoms, and one double bond. Alkynyl radicals normally contain about two to about twenty carbon atoms and one or more, for example two, triple bonds, preferably such as two to ten carbon atoms, and one triple bond. Examples of alkynyl radicals are straight-chain or branched hydrocarbon radicals which contain one or more triple bonds. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3 dimethylbutyl. Both the main chain as well as the branches may furthermore contain heteroatoms as for instance N, O, S, Se or Si or carbon atoms may be replaced by these heteroatoms.

For purposes of the present disclosure, the term “coagulation” or “clotting” refers to a process by which blood changes from a liquid to a gel, forming a blood clot. It potentially results in hemostasis, the cessation of blood loss from a damaged vessel, followed by repair. The mechanism of coagulation may involve activation, adhesion and aggregation of platelets, as well as deposition and maturation of fibrin. In one disclosed embodiment, coagulation may begin almost instantly after an injury to the endothelium lining a blood vessel. Exposure of blood to the subendothelial space may initiate two processes: changes in platelets, and the exposure of subendothelial tissue factor to plasma factor VII, which ultimately may lead to cross-linked fibrin formation. Platelets may immediately form a plug at the site of injury; this is called primary hemostasis. Secondary hemostasis may occur simultaneously: additional coagulation (clotting) factors beyond factor VII may respond in a cascade to form fibrin strands, which strengthen the platelet plug.

For purposes of the present disclosure, the term “heteroatom” as used herein means an atom of any element other than carbon or hydrogen.

For purposes of the present disclosure, the term “gel,” and “nanogel” are used interchangeably. These terms refer to a is a network of polymer chains, entrapping water or other aqueous solutions, such as physiological buffers, of over 99% by weight. In an embodiment of the present disclosure, the polymer chains may be a peptide with repetitive sequences. If the self-assembly of the ultrashort peptides occurs in aqueous solution, hydrogels are formed. If organic solvents are used, organogels are formed.

For purposes of the present disclosure, the term “PBS” refers to a buffer solution commonly used in biological research, which is an abbreviation of phosphate-buffered saline. It is a water-based salt solution, helping to maintain a constant pH, as well as osmolarity and ion concentrations to match those of most cells. In some embodiments, PBS may include a water-based salt solution containing disodium hydrogen phosphate, sodium chloride and, in some formulations, potassium chloride and potassium dihydrogen phosphate.

For purposes of the present disclosure, the term “scaffolds” as used herein means the supramolecular network structures made from self-assembling ultra-short peptide or other polymer materials in the bioinks that provide support for the cellular components.

For purposes of the present disclosure, the term “ultra-short peptide” refers to a sequence containing 3-7 amino acids. The peptides according an aspect of the present disclosure are also particularly useful for formulating aqueous or other solvent compositions, herein also sometimes referred to as “inks” or “bioinks” when mixed with cellular components, which may be used as inks for printing structures and as bioinks for printing cellular or tissue structures, in particular 3D structures. Such printed structures make use of the gelation properties of the peptides according to features of the present disclosure.

For purposes of the present disclosure, the terms “biocompatible” (which also can be referred to as “tissue compatible”) and “biocompatibility”, as used herein, refer to the property of a hydrogel that produces little if any adverse biological response when used in vivo.

For purposes of the present disclosure, the terms “v/v.” “v/v %” and “% v/v” are used interchangeably. These terms refer to Volume concentration of a solution.

For purposes of the present disclosure, the terms “w/v.” “w/v %” and “% w/v” are used interchangeably. These terms refer to Mass concentration of a solution, which is expressed as weight per volume.

### DESCRIPTION

In one embodiment, the peptide-based hydrogels in the present disclosure have the ability to stop bleeding and make a clot.

In one embodiment, the peptide-based hydrogels in the present disclosure are not cytotoxic and have high biocompatibility in vitro.

In one embodiment, the peptide-based hydrogels in the present disclosure have high hemostatic efficiency in vivo.

In one embodiment, the scaffolds are self-assembling nanofibrous ultrashort peptide hydrogels. The present disclosure provides ultrashort peptide sequences containing repetitive sequences capable of forming low molecular weight nanogels by self-assembly, wherein the ultrashort peptides are amphiphilic. The ultrashort peptides are able to self-assemble into supramolecular structures, having a composition of amino acids A, B, X, such as

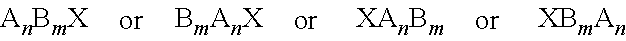

wherein the total number of amino acids of the ultrashort peptide does not exceed 7 amino acids;
wherein A are comprised of aliphatic, i.e. non-aromatic, hydrophobic amino acids, selected from the group of aliphatic amino acids, such as isoleucine and leucine, with n being an integer being selected from 0-5;
wherein B are comprised of one aromatic amino acid, such as tyrosine, tryptophan, or phenylalanine, preferably the hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
wherein X is comprised of a polar amino acid, selected from the group of aspartic acid, glutamic acid, lysine, arginine, histidine, cysteine, serine, threonine, asparagine, and glutamine; and wherein when m=1, n>2.

In a preferred embodiment, the present disclosure provides ultrashort peptide sequences containing repetitive sequences capable of forming low molecular weight nanogels by self-assembly, wherein the ultrashort peptides are amphiphilic. The ultrashort peptides are able to self-assemble into supramolecular structures, having a composition of amino acids A, B, X, such as

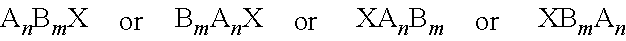

wherein the total number of amino acids of the ultrashort peptide does not exceed 7 amino acids;
wherein A are comprised of aliphatic, i.e. non-aromatic, hydrophobic amino acids, selected from the group of aliphatic amino acids, such as isoleucine and leucine, with n being an integer being selected from 2-5;
wherein B are comprised of one aromatic amino acid, such as tyrosine, tryptophan, or phenylalanine, preferably the hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 1 and 2; and
wherein X is comprised of a polar amino acid, selected from the group of aspartic acid, glutamic acid, lysine, arginine, histidine, cysteine, serine, threonine, asparagine, and glutamine.

The amphiphilic peptide sequences containing repetitive sequences provided in the present disclosure show true supergelating properties, forming low molecular weight nanogels by entrapping water or other aqueous solutions, such as physiological buffers, of over 99% by weight. Therefore, hydrogels can be generated. These amphiphilic peptides have an innate propensity to self-assemble to 3D fibrous networks in form of hydrogels. These gels can also be termed nanogels, because the diameter of the single fibers of the gel's fiber network have nanometer diameters. These peptide compounds are self-driven by non-covalent interactions to form soft solid material. Based on the nature of the peptides involved, generally composed of natural amino acids, these soft materials can easily be used for biomedical applications, for tissue engineering, but also for technical applications.

It should be appreciated that the novel peptides have introduced aromatic amino acids in the hydrophobic part of the amphiphilic peptide structure. This is a significant improvement over prior peptides, which focus solely on peptides containing aliphatic amino acids. The inclusion of aromatic amino acids is crucial for improving the self-assembly process over prior peptide configurations such as disclosed in WO 2011/123061 A1 which is incorporated herein by reference.

It should be appreciated that the novel peptides do not focus on the orientation of the hydrophobic part of the peptide compound as being limited to the N-terminus and the polar hydrophilic part limited to the C-terminus as is the case in prior peptides. The present amphiphilic peptides work well with having both orientations, as of N-terminus-hydrophobic part-hydrophilic part-C-terminus as well as N-terminus-hydrophili part-hydrophobi part-C-terminus.

Factors that Affect Peptide Self-Assembly: Concentration and Temperature

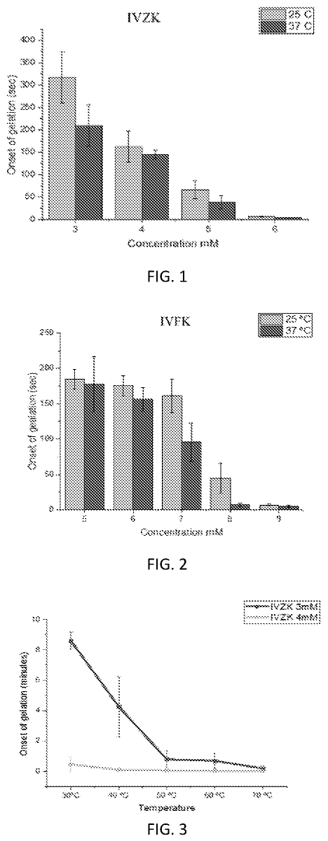

In one embodiment, the nanofiber formation of the self-assembling peptides is affected by concentration of the peptide solution (Luo, Z., S. Wang, and S. Zhang, Fabrication of self-assembling D-form peptide nanofiber scaffold d-EAK16 for rapid hemostasis. Biomaterials, 2011. 32(8): p. 2013-2020). The gelation time of different peptides at various concentrations was recorded using a coagulation meter. FIGS. 1 and 2 illustrate the gelation time of two peptides with different concentrations at two different temperatures (25° C., 37° C.). As shown in both FIGS. 1 and 2, as the concentration of peptide increases, the required time for gelation decreases.

In one embodiment, a higher concentration of peptides in solution produces a higher density of nanofibers, thus promoting hydrogel formation. These highly organized nanofibers are hydrated as water molecules fit through the nanopores. Similar observations were made with other self-assembling peptides such as RADA16 (Luo, Z., S. Wang, and S. Zhang, Fabrication of self-assembling D-form peptide nanofiber scaffold d-EAK16 for rapid hemostasis. Biomaterials, 2011. 32(8): p. 2013-2020; Yokoi, H., T. Kinoshita, and S. Zhang, Dynamic reassembly of peptide RADA16 nanofiber scaffold. Proceedings of the National Academy of Sciences of the United States of America, 2005. 102(24): p. 8414-8419).

In one embodiment, the peptide gelation is impacted by temperature. As shown in FIGS. 1 and 2, peptides IVZK (SEQ ID NO. 2) and IVFK (SEQ ID NO. 1) achieved faster gelation at 37° C., than at 25ºC. This effect can be explained by the increasing of kinetic energy which is helps in bond formation within molecules enabling the peptide solution to form gels faster at 37° C. than at 25° C.

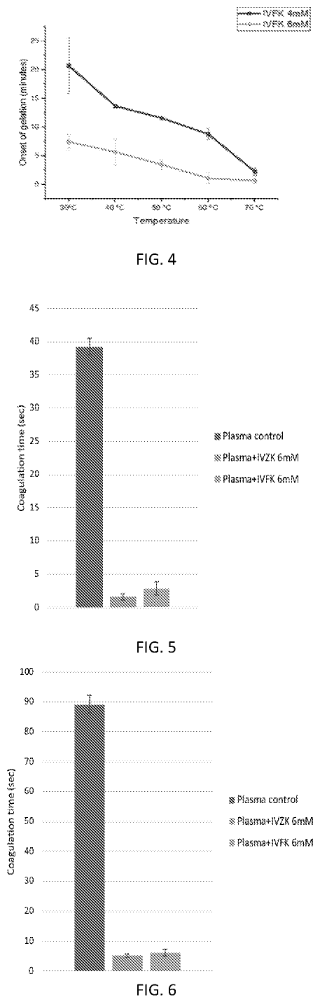

In one embodiment, the gelation time is impacted by temperature. As shown in FIGS. 3 and 4, the onset of gelation occurs faster with the increase of temperature for both IVZK (SEQ ID NO. 2) and IVFK (SEQ ID NO. 1) at different concentrations.

### In Vitro Effect of Self-Assembling Nanofiber Scaffolds for Enhancing Hemostasis

In one embodiment, the ultra-short peptides can self-assemble into nanofibers, which promotes coagulation. The ability of these peptides to enhance hemostasis through coagulation might be due to their ability to self-assemble into nanofibers. This property of the self-assembling ultra-short peptides offers great advantages over the other biomaterials available in the clinics such as chitosan. These advantages include: highly cost-effective due to it extremely short sequence; facile and quick synthesis; easy handling due to its simple way of self-assembly; no toxicity and low immunogenicity.

In one embodiment, the peptides have short gelation time, which makes them especially useful as the adhesive sealant in hemostatic applications where the gel must form rapidly, remain at the site of administration, and not dilute with the passage of fluid (Nie, W., et al., Rapidly in situ forming chitosan e-polylysine hydrogels for adhesive sealants and hemostatic materials. Carbohydrate polymers, 2013. 96(1): p. 342-348). The formation of fibers (gelation) to the sufficient extent to stop bleeding and hold the blood cells in the networks of these fibers on a short time scale is needed to meet the requirements of tissue sealants and hemostatic materials (Lih, E., et al., Rapidly curable chitosan PEG hydrogels as tissue adhesives for hemostasis and wound healing. Acta biomaterialia, 2012. 8(9): p. 3261-3269). The effect of the self-assembling property of the peptides in enhancing hemostasis can be impacted by the following factors.

### In Vitro Effect of Self-Assembling Nanofiber Scaffolds for Enhancing Hemostasis: Coagulation Time

The blood coagulation is a process that results in the formation of fibrin as the end product of the plasma coagulation cascade. This cascade includes plasma proteins known as coagulation factors. The managed interaction between platelets, leukocytes, endothelial cells, and plasma coagulation proteins is essential to maintain hemostasis and to stop bleeding. Inactivation of one or more of these components may lead to pathological blood coagulation and life-threatening conditions such as consumptive coagulopathy or disseminated intravascular coagulation (DIC). In contrast, unintended inhibition of the coagulation pathways may lead to hemorrhage (Potter, T. M., et al., In Vitro Assessment of Nanoparticle Effects on Blood Coagulation, in Characterization of Nanoparticles Intended for Drug Delivery. 2018, Springer. p. 103-124).

In one embodiment, the peptide hydrogels reduce blood coagulation time.

In one embodiment, an application of the peptide-based hemostatic material in the present disclosure to the bleeding wound produces a coagulation time of less than 40 seconds. The coagulation time of normal plasma (without self-assembling peptides) and plasma with IVZK (SEQ ID NO. 2) or IVFK (SEQ ID NO. 1) hydrogels as initiators for clot formation was compared using a partial thromboplastin time (aPTT) test. The hemostatic properties of the peptides were evaluated by measuring the coagulation time after mixing the peptide solution with plasma. FIG. 5 shows the comparison of coagulation time of plasma with and without self-assembling peptides. The coagulation time of the control (without self-assembling peptides) was around 40 seconds, which is within the normal range. However, after the addition of peptides solution, the clotting time was dramatically decreased to around 5 seconds.

In another embodiment, the coagulation time of plasma with deficiencies in clotting factors was also reduced with the addition of self-assembling peptides, as shown in FIG. 6.

A chitosan/dopamine/diatom-biosilica composite beads (CDDs) was developed that were known to exhibit the shortest clotting time, around 1.35 min (Wang, Y., et al., Multifunctional chitosan/dopamine/diatom-biosilica composite beads for rapid blood coagulation. Carbohydrate polymers, 2018. 200: p. 6-14). Chitosan, a muco-adhesive polymer with hemostatic properties independent of host coagulation mechanisms, has also been used and accelerated coagulation of blood in vitro from 5 to 2 min as a gel (Rao, S. B. and C. P. Sharma, Use of chitosan as a biomaterial: studies on its safety and hemostatic potential. Journal of Biomedical Materials Research: An Official Journal of The Society for Biomaterials and The Japanese Society for Biomaterials, 1997. 34(1): p. 21-28).

In one embodiment, the self-assembly peptides in the present disclosure have the ability to coagulate blood within a time frame of just seconds, which is the fastest of all known hemostatic materials.

It is also known that ions have an important role in self-assembly processes. Since accelerating hemostasis is an important clinical applications of the self-assembling peptide scaffolds, and because the blood contains a plethora of ions, it is possible that upon contact with blood, the ions within the environment can trigger rapid self-assembly of the peptide into nanofibers to form the hydrogel (Luo, Z., S. Wang, and S. Zhang, Fabrication of self-assembling D-form peptide nanofiber scaffold d-EAK16 for rapid hemostasis. Biomaterials, 2011. 32(8): p. 2013-2020). Another possibility is that the positive charge of these peptides may promote binding with negatively charged proteins in plasma through intermolecular coulombs interactions, thus resulting in the formation of a nanofiber clot.

### In Vitro Effect of Self-Assembling Nanofiber Scaffolds for Enhancing Hemostasis: Adhesion of Erythrocytes in Peptide Scaffolds

The ability of self-assembling peptides to shorten blood coagulation time has been thought to occur through nanofiber entanglements that trap blood components (Luo, Z., S. Wang, and S. Zhang, Fabrication of self-assembling D-form peptide nanofiber scaffold d-EAK16 for rapid hemostasis. Biomaterials, 2011. 32(8): p. 2013-2020).

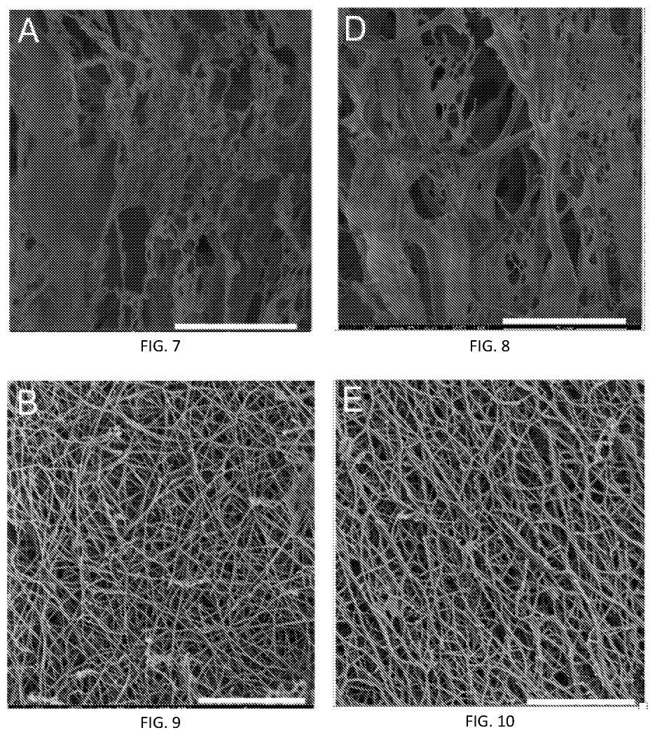

In one embodiment, the self-assembling peptides form a dense network of highly entangled nanofibers as shown in FIGS. 7-12. The morphological characteristics of the nanofibers and the surface adhesion of blood cells were characterized using (SEM). The scale bar in FIGS. 7-12 is 3 μm. FIGS. 7-10 show the morphology of the nanofibers of IVFK (SEQ ID NO. 1) and IVZK (SEQ ID NO. 2) hydrogels in water and PBS.

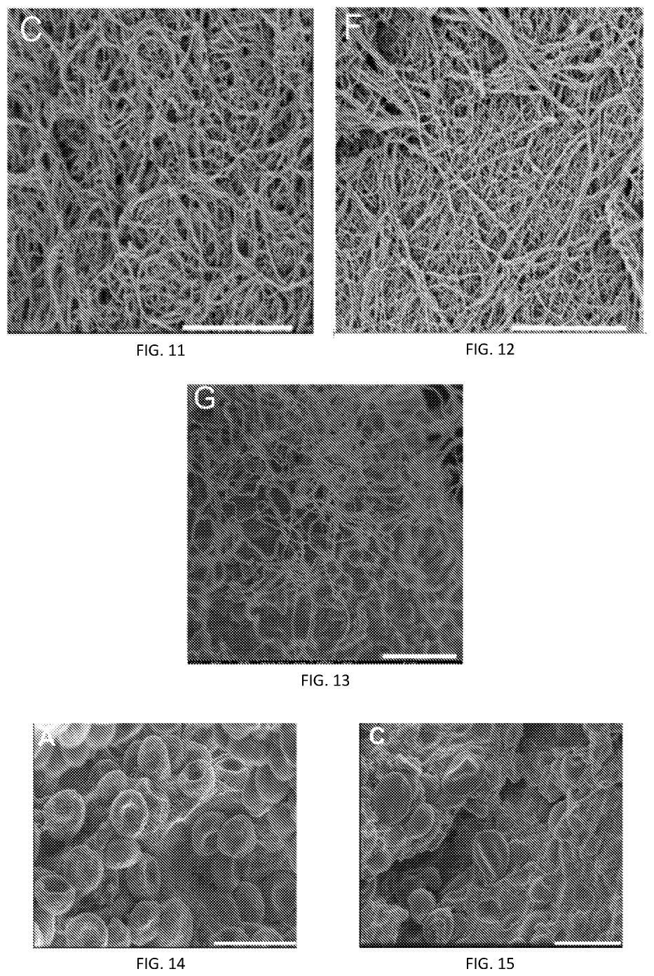

In another embodiment, mixing of the peptide solution with uncoagulated plasma formed clots directly without the addition of any coagulation initiator. As shown in FIGS. 11 and 12, nanofiber formation by peptides mixed with uncoagulated plasma is similar to the fibrin shown in FIG. 13. The scale bar in FIG. 13 is also 3 μm.

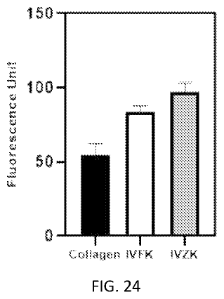

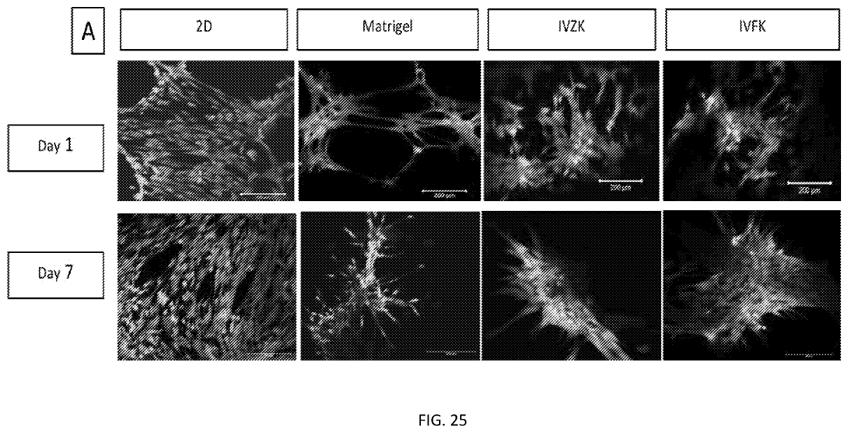

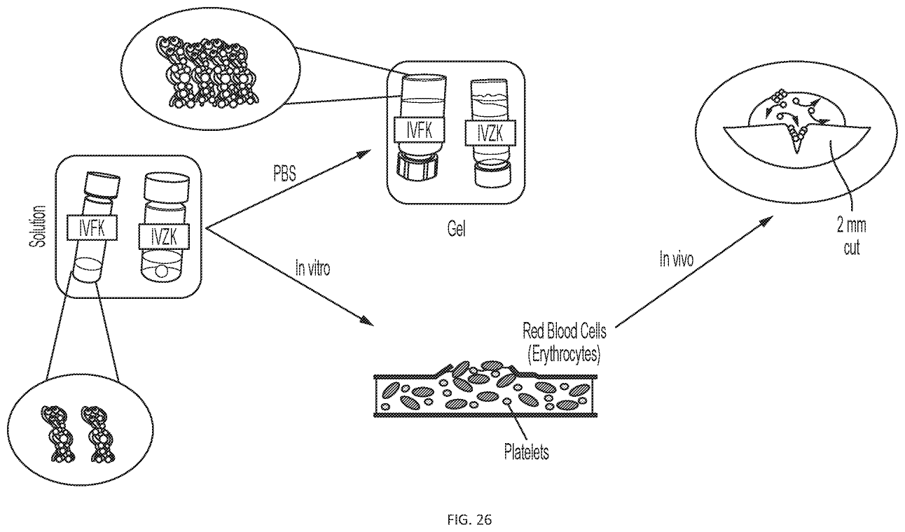

In one embodiment, the self-assembling peptides assemble into nanofibers and trap blood cells of anticoagulated blood. In the present disclosure, the anticoagulated blood was mixed with peptide solution to confirm the nanofiber formation of hydrogels, which is capable of entrapping blood components (Luo, Z., S. Wang, and S. Zhang, Fabrication of self-assembling D-form peptide nanofiber scaffold d-EAK16 for rapid hemostasis. Biomaterials, 2011. 32(8): p. 2013-2020). As shown in FIGS. 24-17, the peptide solution formed a dense fiber network that captured large amounts of blood cells and locked them in, which is an excellent indicator of hemostasis.

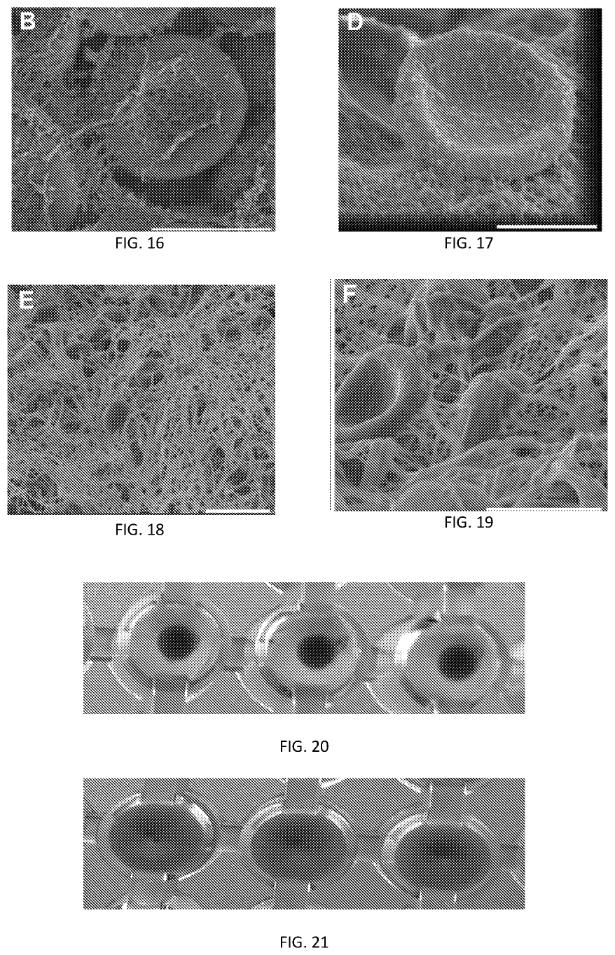

In one embodiment, this nanofiber-based clot appears to have the ability to hold blood cells, which are normally trapped by polymerized fibrin during the natural coagulation process. The blood cells trapped by polymerized fibrin during the natural coagulation process are shown in FIGS. 18 and 19. The scale bars in FIGS. 14, 15 and 18 are 10 μm, while the scale bars in FIGS. 16, 17 and 19 are 5 μm.

Because the formation of nanofiber clots by self-assembling peptides occurs under anticoagulating conditions, the peptides may provide significant hemostatic activity even under coagulopathic conditions.

There are many hemostatic materials that have shown the potential to enhance the body's natural clotting mechanisms. Hemostatic topical products based on biomacromolecules and proteins, such as chitosan and fibrinogen, or inorganic materials, such as kaolin, are able to facilitate fibrin nanofiber formation. However, there are several drawbacks to the use of these materials, including limited efficacy, difficulty to use (e.g., very viscous gel or powder), generation of heat on contact with blood, and lack of a biodegradable mechanism thus requiring surgical removal after use (Gordy, S. D., P. Rhee, and M. A. Schreiber, Military applications of novel hemostatic devices. Expert review of medical devices, 2011. 8(1): p. 41-47). On the other hand, the use of peptide hydrogels to enhance coagulation by generating nanofibers have many advantages. These include their very fast activity to form clots just seconds after application, their lack of cytotoxicity, low cost, and easy preparation and synthesis.

### In Vitro Effect of Self-Assembling Nanofiber Scaffolds for Enhancing Hemostasis: Agglutination In Vitro

In one embodiment, the self-assembling peptides demonstrate agglutination activity. An in vitro assay was performed for nanofiber formation that has previously been correlated to in vivo hemostatic activity (Luo, Z., S. Wang, and S. Zhang, Fabrication of self-assembling D-form peptide nanofiber scaffold d-EAK16 for rapid hemostasis. Biomaterials, 2011. 32(8): p. 2013-2020). Ions and salts in the solution of red blood cells may have an important effect on peptide self-assembly into nanofibers (Luo, Z., S. Wang, and S. Zhang, Fabrication of self-assembling D-form peptide nanofiber scaffold d-EAK16 for rapid hemostasis. Biomaterials, 2011. 32(8): p. 2013-2020). The effect of self-assembling peptides in the solution of red blood cells was tested by mixing these peptides with red blood cells (RBCs) in a 96-well microtiter plate with V-shaped wells and allowing the RBCs to settle. As shown in FIGS. 20-22, the peptides formed nanofibers, keeping RBCs in suspension in a manner indicative of agglutination. However, no agglutination appears in the wells of without peptides (the negative control).

These observations are consistent with a report that other peptides capable of self-assembling into nanofibers demonstrated agglutination activity in solution with red blood cells (Luo, Z., S. Wang, and S. Zhang, Fabrication of self-assembling D-form peptide nanofiber scaffold d-EAK16 for rapid hemostasis. Biomaterials, 2011. 32(8): p. 2013-2020). However, the self-assembling ultra-short peptides in the present disclosure offers great advantages over the previously reported peptides. These advantages include: highly cost-effective due to it extremely short sequence; facile and quick synthesis; very easy to handle due to its simple way of self-assembly; no toxicity and low immunogenicity.

### In Vitro Effect of Self-Assembling Nanofiber Scaffolds for Enhancing Hemostasis: Adhesion of Platelets

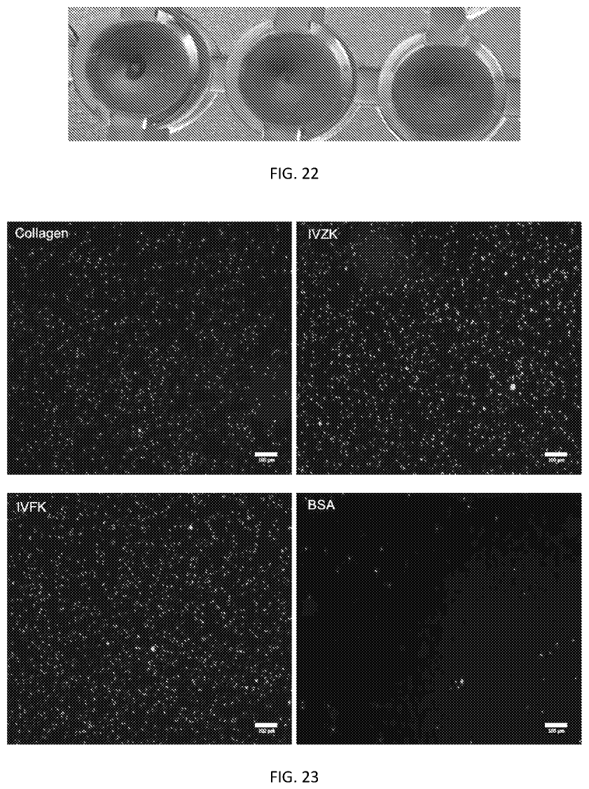

In one embodiment, the self-assembling peptides induce platelet adhenrance. In a preferred embodiment, the self-assembling peptides induce more platelet adhenrance than collagen. One important factor for accelerating blood coagulation could be by promoting the recruitment and adherence of platelets to the site of injury as these processes play a critical role in primary hemostasis (O'Shaughnessy, D. and R. Gill, 19 Cardiothoracic surgery. Practical Hemostasis and Thrombosis, 2009: p. 194). Therefore, platelet adherence induced by peptide hydrogels, type I collagen (positive control), and bovine serum albumin (BSA) (negative comtrol) was tested and compared. As shown in FIG. 23, considerable platelet adhesion was induced by IVZK (SEQ ID NO. 2) and IVFK (SEQ ID NO. 1) hydrogels. The scale bar in FIG. 23 is 100 μm. The level of platelet adhesion induced by IVZK (SEQ ID NO. 2) and IVFK (SEQ ID NO. 1) hydrogels was comparable to that on the surface of collagen, the positive control. The level of platelet adhesion induced by peptide hydrogels, type I collagen (positive control) was also quantified, as shown in FIG. 24. According to FIG. 24, the level of platelet adhesion induced by peptide hydrogels is even higher than that of collagen. However, little platelet adhesion was induced by BSA, the negative control. Therefore, our peptides induced platelet adhesion, a process that may help trigger the coagulation system.

### Biocompatibility Assays

An ideal biomaterial should be biocompatible and non-toxic, which could be evaluated through in vitro cytotoxicity assays. In one embodiment, the self-assembling peptides in the present disclosure are biocompatible and non-toxic. Therefore, the biocompatibility and cytotoxicity of the peptide hydrogels in the present disclosure were tested. Live/dead® staining was performed after three and seven days of culture to assess the cytocompatibility with BM-MSC. The Live/dead® staining results are shown in FIG. 25, in which live cells are stained in green with Calcein AM and dead cells in red with ethidium homodimer-1. Number of cells cultured with peptide hydrogels increased with the increase of culture time, and few dead cells was found in the analyzed fields.

### Point-of-Care 3D Printing Pen

The point-of-care 3D printing pen is a multi-purpose do-it-yourself device. 3D printing has revolutionized the way of production, making it cheap, personalized and manufacturable on-site. The global 3D printing market is estimated to reach a size of 35.3B USD by 2027. It is expected to witness a Compound Annual Growth Rate (CAGR) of 14.6% over the forecast period. While the technology is affordable for standard consumer, the need of portability has soon moved the printer on from a bulky device into the hand-held 3D printers and printing pens, which allow using the fine motor skills of human hand to create tailored 3D structures. Architects, designers, and hobbyists at present, compose most of this market. Recently, 3D bioprinting, which is the printing of cell-laden materials for the manufacturing of human tissue, has become portable, when surgeons hand-draw new cells directly onto bone in the middle of a surgical procedure. According to an industry research, the global 3D Stereoscopic Drawing Doodling Printing Pen market size is projected to reach USD 88 million by 2026, from USD 51 million in 2020, at a CAGR of 9.5% during 2021-2026. The key revolutions around 3D printing are now to make the technique portable and to be compatible with a wider range of materials that can be printed. Portability gives access to the technology even in remote areas or challenging situations. Therefore, a hand-held printer is able to become a daily-use product for most of the consumers.

Conventionally, 3D printing is conducted by the use of robotic printers, suitable in-house designed nozzles, additive manufacturing printers and nozzles, and electronic engineering tools and software. Some examples of conventional 3D bioprinters are robotic arm printers (Dobot), an in-house developed dual-arm 3D bioprinter (Yaskawa) with 6 axes of freedom on each arm, which increases flexibility and doubles the workspace of common 3D bioprinters, and a GeSIM printer provided recently by the GeSIM company (BS3.2). Dual- and triaxial nozzles are also developed in house and by the company GeSIM to fit the characteristics of self-assemble ultrashort peptide inks disclosed in the present disclosure. The 3D printing systems comprises a microfluidics-based extrusion unit, which gradually mixes three solutions together in the custom-made coaxial nozzle to form peptide bioink instantaneously. In some instance, the Graphical User Interface (GUI) is implemented to provide the user with accessibility to set parameters for printing, pumping, and temperature control.

It has been shown that dual-arm 3D printer is able to enhance the resolution of 3D structures both of self-assembly peptide inks and cell-laden peptide bioinks in the present disclosure, during and after the printing process. The structure fidelity was assessed, and the instantaneous gelation of the peptide in the extrusion unit made for a consistent and smooth extrusion that produced firm and stable constructs. Moreover, the custom nozzle embedded in the above-mentioned 3D bioprinter was found to be compatible with other commercial peptide bioinks such as the Biogelx™ peptide. The results were compared to commercial 3D bioprinters and were found to be consistent.

In one embodiment, these keypoints of the above-mentioned bioprinters can be transformed into a hand-held 3D printing pen.

In a preferred embodiment, the hand-held 3D printing pen uses peptide-based material as ink.

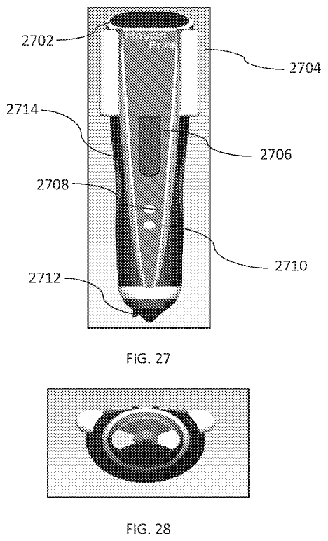

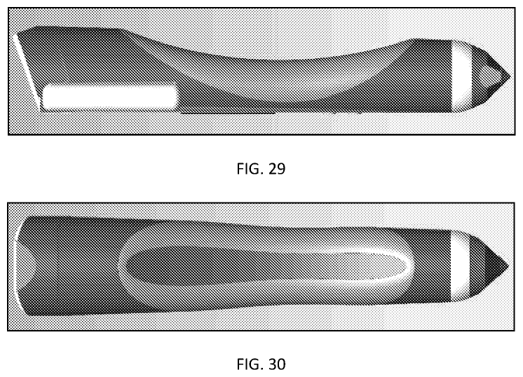

In one embodiment, nozzles, cartridges or eventual tubing, temperature control, flow-rate controls and actuators, and batteries are enclosed in a portable pen-like housing. An exterior design of the portable 3D printer/hand-held 3D printing pen is shown in FIG. 27. As shown in FIG. 27, the portable 3D printer/hand-held 3D printing pen comprises a body of the device (2714), a solar power panel (2702), two bioink compartments located on both sides of the device (2704) that are reloadable and interchangeable, a screen (2706) in the front of the device showing various parameters such as temperature, flow rate, etc., a stop button (2708), a start button (2710), and a thermal heating and cooling nozzle (2712). A bottom view, a side view and a back view of the device are shown in FIGS. 28-30, respectively. The Printing Pen is fed with a two-component bioink cartridge, which carries the peptide hydrogel and activation fluid (saline buffers) in separate channels. Once the user activates the pen, two screw-driven pistons gradually pump the bioink components into the nozzle to begin gelation. As the bioink forms, a consistent thread of gel is extrude from the nozzle outlet at the tip of the pen. A start button and a stop button give the user the option to pause extrusion when desired.

In a preferred embodiment, an increase flow button and decrease flow button allow the user to vary the flow rate within a set range to speed up or slow down gelation.

In one embodiment, the portable 3D printer/hand-held 3D printing pen will also have the capacity to bioprint cells using the printing pen.

In one embodiment, the nozzle and ink cartridges adopting a modular designs are incorporated for application-specific requirements. In the design of this device, the requirements of commonly used bioinks in terms of temperature and extrusion speed control are considered.

In one embodiment, the heads and nozzles are interchangeable, making the pen adaptable for a broader range of applications. This broadens the range of possible functions, including extrusion, spraying, and quick application.

In one embodiment, the device has the capability of thermal heating and cooling the nozzle, making the 3D printing pen compatible with a wider variety of bioinks.

In one embodiment, additional sensor feedback for flow, temperature and control are included in the 3D printing pen to enhance accuracy and reliability.

In another embodiment, a low-intensity UV light is added to the tip at the bottom of the 3D printing pen.

In one embodiment, the 3D printing pen is waterproofing and is suitable for underwater applications.

In one embodiment, the 3D printing pen runs on solar power, making it easier to use in remote locations, given that disaster relief and first-aid care are also potential applications. In another embodiment, the 3D printing pen also has a battery-powered backup system for reliability.

In one embodiment, the 3D printing pen has a camera lens at the tip of the pen for real-time monitoring of ink extrusion. This could be streamed in real-time over Wi-Fi or Bluetooth to the user's smartphone through an integrated software application.

In one embodiment, the 3D printing pen is designed to have the flexibility to add various tubings to the cartridges could expand ink supply from external reservoirs in cases where larger volumes are needed.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### EXAMPLES

### Example 1

### Materials

The tetrameric self-assembling peptides were custom synthesized by Bachem® AG, (Budendorf, Switzerland). Bone marrow-derived human mesenchymal stem cells (hMSCs, PT-2501) were purchased from Lonza®, USA. Cells were cultured in medium (PT-4106E Lonza® USA) and supplemented with mesenchymal cell growth supplements (PT-4106E Lonza®. USA), Gentamicin Sulfate Amphotericin-B (PT-4501E Lonza®, USA) and with L-Glutamine (PT-4107E Lonza®, USA). T175 or T75 cell culture flasks and 96 and 48 well-plates were ordered from Corning®, USA. CytoTox 96® Non-Radioactive Cytotoxicity were purchased from Thermo Fisher Scientific®, USA, and Promega®, USA respectively. The CellTiter-Glo® luminescent 3D cell viability assay was purchased from Promega®, USA.

### Example 2

### Peptides Preparation

The tetramer in the form of lyophilized powder were dissolved in Milli-Q® water. Then, 10× phosphate buffered saline (PBS) was added to the aqueous peptide solution at a final volume ratio of peptide solution to 10×PBS of 9:1. The gelation of all peptides occurred within a few minutes.

### Example 3

### Determination of Gelation Time by Coagulation Meter

In this study, onset of gelation was assessed using coagulation meter MC 10 PLUS (MERLIN® Medical, Germany). The peptide solution at different concentrations was used and checked under various conditions. For the coagulation experiment, 300 μl of peptide solution was added into a tube and placed in the preheated position for 50 seconds before adding 30 μl of PBS. The time at which the gel formed was then recorded. The temperature was chosen in advance and all solutions were incubated at the desired temperature before conducting the measurements.

### Example 4

### Scanning Electron Microscopy (SEM) Characterization of Assembled Nanofibers

Peptide hydrogels were characterized using SEM to visualize the morphology of the nanofibers. The hydrogel samples were placed on 18×18 mm glass cover slips and were left to solidify for 10-20 minutes. Then, the hydrogel samples were dehydrated by gradually immersing in increasing concentrations of 20%, 40%, 60%, 80%, and 100% (v/v) ethanol solutions for 5 min in each solution. Further dehydration in 100% ethanol solution was continued by changing the 100% ethanol solution with a fresh one twice for 5 min each followed by a third time for 2 hours. The dehydrated samples were subsequently placed into a critical point dryer for evaporation before being mounted onto aluminum SEM pin stubs with double-stick conductive carbon tape. A final sputter coating of 10 nm thick layer of iridium was performed prior to imaging with FEI® Magellan™ XHR. Biological peptide hydrogel coating specimen were fixed with 2.5% glutaraldehyde (diluted from 25%) in water for overnight 4° C., the post fixation was done by fixing in 1% osmium teteroxide in 0.1 M PBS for one hour in the dark followed by washing 3 times by ddH₂O. Then, serial dehydration was conducted in 10 mL of H₂O (twice), 25% ethanol, 50% ethanol, 75% ethanol, 80% ethanol, 90% ethanol, and 100% ethanol (twice) in sequence. Samples in ethanol were then critically point dried using liquid CO₂ (Sorvall® Critical Point Drying System). The samples were ready to image.

### Example 5

### Nanofiber Activity In Vitro

The ability of the two peptides to form a nanofiber-based clot in vitro was determined using an assay similar to the one previously described (Luo, Z., S. Wang, and S. Zhang, Fabrication of self-assembling D-form peptide nanofiber scaffold d-EAK16 for rapid hemostasis. Biomaterials, 2011. 32(8): p. 2013-2020). Briefly, 95 μL of peptide solution were added to V-shaped 96-well microtiter plates followed by the addition of 10 μL of 10% rabbit red blood cells (RBCs). The wells were sealed with an optically clear adhesive film, agitated at ˜900 rpm for 15 min, and then incubated at 4° C. for at least 4 h prior to examination.

### Example 6

Blood Coagulation Test (aPTT)

The aPTT test was performed in a coagulation meter MC 10 PLUS (MERLIN® Medical, Germany). First, 100 μL of plasma was incubated at 37° C., followed by adding 100 μL of a-PTT reagent. Then, the plasma-reagent mixture was incubated at 37° C. for 5 minutes (activation time). Finally, 100 μL of pre-warmed 0.025 mM calcium chloride was added and time clot formation was recorded. In experimental group, 150 μL of plasma was pre-warmed and then 150 μL of peptide solution was added and the time was recorded. Each test was conducted at least three times.

### Example 7

### Surface Coating and Platelet Isolation and Adhesion

The procedure was done as described previously (Chen, C., et al., Hydrogelation of the short self-assembling peptide 13QGK regulated by transglutaminase and use for rapid hemostasis. ACS applied materials & interfaces, 2016. 8(28): p. 17833-17841). Briefly, for surface coating. 200 μL of 6 mM IVFK (SEQ ID NO. 1), 6 mM IVZK (SEQ ID NO. 2), 0.5% (w/v), BSA (PBS, pH 7.4), and 0.01 mg/mL type I collagen (PBS, pH 7.4) solutions were added to the wells of a 96-well black plate and incubated at 4° C. for 24 h. The wells were washed three times with PBS after removing these solutions. Human platelets were isolated from whole human blood containing anticoagulant sodium citrate (0.38%, w/v) as described previously(Chen, C., et al., Hydrogelation of the short self-assembling peptide 13QGK regulated by transglutaminase and use for rapid hemostasis. ACS applied materials & interfaces, 2016. 8(28): p. 17833-17841). The blood collection was approved by IBEC (18IBEC143). Briefly, platelet-rich plasma (PRP) was collected by centrifugation at 200×g for 20 min at 25° C. Then, PRP was diluted at a 1:1 ratio with PBS and subjected to second centrifugation at 800×g for 12 min at 25° C. The isolated platelets were washed three times with Tyrode's buffer (Hepes 20 mM, glucose 137 mM, BSA 1 mg/mL, NaH₂PO₄ 3.3 mM, NaCl 137 mM, KCl 2.7 mM), followed by staining with calcein-AM (1 μg/mL) for 30 min in the dark at room temperature to stained platelets. After washing twice with PBS, the stained platelets were added into the coated wells and incubated for 1 h at 37° C. Subsequently, the supernatants were removed, and the unbound platelets were discarded by washing three times with PBS. The fluorescence of adherent platelets was measured by fluorescence microscopy.

### Example 8

### Hemolytic Activity

The hemolytic activity of peptide solution was assessed using fresh human red blood cells. Specifically, RBCs were collected by centrifugation (1000×g. 5 min) of the whole blood from healthy volunteers and washed three times with 1×PBS. 100 μL of 8% (v/v) RBCs solution was mixed with 100 μL of different concentrations of peptide solutions in a sterile 96-well plate and incubated for 1 h at 37° C. Then, the 96-well plate was centrifugated at 1000×g for 10 min, and 100 μL of the supernatant was transferred into a new 96-well plate. Finally, the hemoglobin release was measured by recording the absorbance at 540 nm (A540) using the plate reader (PHERAstar® FS, Germany). Hemoglobin release in PBS was used as negative control for 0% release and in 0.1% (v/v) Triton X-100 as positive control for 100% release.

### Example 9

### Cell Culture of Human Bone Marrow Mesenchymal Stem Cells (BM-MSCs)

Human bone marrow mesenchymal stem cells, (hMSCs, PT-2501) were purchased from Lonza®, USA. Cells were cultured in medium (PT-4106E Lonza®, USA) and supplemented with mesenchymal cell growth supplements (PT-4106E Lonza®, USA), Gentamicin Sulfate Amphotericin-B (PT-4501E Lonza®, USA) and with L-Glutamine (PT-4107E Lonza®, USA). The cells were maintained either in T75 or T150 cell culture flasks (Corning®, USA) at 37° C. in a humidified incubator with 95% air and 5% CO₂. The cells were subcultured by using trypsin at approximately 80% confluence. The culture media was changed every 2-3 days.

### Example 10

### Live/Dead Staining and Cytotoxicity Assay

The MSCs cells were incapsulated with peptides solution according to the protocol described above. After one, three and seven days of incubation, the spent media was removed and replaced with 1×PBS solution containing approximately 2 mM calcein AM and 4 mM ethidium homodimer-1 (LIVE/DEAD® Viability/Cytotoxicity Kit, Life Technologies™) and incubated for 40 min in the dark. Before imaging, the staining solution was removed, and fresh PBS was added. Stained cells were imaged under an inverted confocal microscope (Zeiss® LSM 710 Inverted Confocal Microscope, Germany).

cytotoxicity assay was performed using CytoTox 96® Non-Radioactive Cytotoxicity Assay according to the manufacturer's protocol. In brief, 50 μl of media was removed from each sample and mixed with 50 μl of CytoTox 96® Reagent, incubated for 30 minutes in the dark. Then, 50 μl of stop solution was added. Finally, the absorbance was measured at 490 nm using a 96-well plate reader (PHERAstar® FS, Germany).

### Example 11

### 3D Cell Proliferation Assay

The CellTiter-Glo® luminescent 3D cell viability assay was used to determine proliferation of cells in hydrogels based on quantification of the produced ATP, which points to the presence of metabolically active cells (Gilbert, D. F., et al., A novel multiplex cell viability assay for high-throughput RNAi screening. PLOS One, 2011. 6(12): p. e28338). At each time point, the kit was equilibrated at room temperature for approximately 30 minutes. CellTiter-Glo® Reagent equal to the volume of cell culture medium present in each well was added. The mixture was mixed for 5 minutes to decompose the hydrogels and then incubated for 30 minutes. After incubation, the luminescence was recorded using a plate reader (PHERAstar® FS, Germany).

It is intended that the invention not be limited to the particular embodiment disclosed herein contemplated for carrying out this invention, but that the invention will include all embodiments falling within the scope of the claims.

All documents, patents, journal articles and other materials cited in the present application are incorporated herein by reference.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

### REFERENCES

The following references are referred to above and are incorporated herein by reference:
1. Hsu, B. B., et al., Clotting mimicry from robust hemostatic bandages based on self-assembling peptides. ACS nano, 2015. 9(9): p. 9394-9406.
2. Song, H., L. Zhang, and X. Zhao, Hemostatic efficacy of biological self-assembling peptide nanofibers in a rat kidney model. Macromolecular bioscience, 2010. 10(1): p. 33-39.
3. Kheirabadi, B., Evaluation of topical hemostatic agents for combat wound treatment. US Army Medical Department Journal, 2011.
4. Holcomb, J. B., et al., Implications of new dry fibrin sealant technology for trauma surgery. Surgical Clinics, 1997. 77(4): p. 943-952.
5. Albala, D. M., Fibrin sealants in clinical practice. Cardiovascular surgery, 2003. 11: p. 5-11.
6. Plaisier, B. R. Surgical perspectives to control bleeding in trauma. in Seminars in Anesthesia, Perioperative Medicine and Pain. 2001. Elsevier.
7. Kheirabadi, B. S., et al., Comparative study of the efficacy of the common topical hemostatic agents with fibrin sealant in a rabbit aortic anastomosis model. Journal of Surgical Research, 2002. 106(1): p. 99-107.
8. Cole, D. J., et al., A pilot study evaluating the efficacy of a fully acetylated poly-N-acetyl glucosamine membrane formulation as a topical hemostatic agent. Surgery, 1999. 126(3): p. 510-517.
9. Tuthill, D. D., et al., Assessment of topical hemostats in a renal hemorrhage model in heparinized rats. Journal of Surgical Research, 2001. 95(2): p. 126-132.
10. Crescenzi, V., et al., Synthesis and partial characterization of hydrogels obtained via glutaraldehyde crosslinking of acetylated chitosan and of hyaluronan derivatives. Biomacromolecules, 2003. 4(4): p. 1045-1054.
11. Loo, Y., et al., Peptide bioink: self-assembling nanofibrous scaffolds for three-dimensional organotypic cultures. Nano letters, 2015. 15(10): p. 6919-6925.
12. Ellis-Behnke, R., At the nanoscale: nanohemostat, a new class of hemostatic agent. Wiley interdisciplinary reviews: nanomedicine and nanobiotechnology, 2011. 3(1): p. 70-78.
13. Luo, Z., S. Wang, and S. Zhang, Fabrication of self-assembling D-form peptide nanofiber scaffold d-EAK16 for rapid hemostasis. Biomaterials, 2011. 32(8): p. 2013-2020.
14. Chen, C., et al., Hydrogelation of the short self-assembling peptide 13QGK regulated by transglutaminase and use for rapid hemostasis. ACS applied materials & interfaces, 2016. 8(28): p. 17833-17841.
15. Gilbert, D. F., et al., A novel multiplex cell viability assay for high-throughput RNAi screening. PLOS One, 2011. 6(12): p. e28338.
16. Yokoi, H., T. Kinoshita, and S. Zhang, Dynamic reassembly of peptide RADA16 nanofiber scaffold. Proceedings of the National Academy of Sciences of the United States of America, 2005. 102(24): p. 8414-8419.
17. Nie, W., et al., Rapidly in situ forming chitosan e-polylysine hydrogels for adhesive sealants and hemostatic materials. Carbohydrate polymers, 2013. 96(1): p. 342-348.
18. Lih, E., et al., Rapidly curable chitosan-PEG hydrogels as tissue adhesives for hemostasis and wound healing. Acta biomaterialia, 2012. 8(9): p. 3261-3269.
19. Potter, T. M., et al., In Vitro Assessment of Nanoparticle Effects on Blood Coagulation, in Characterization of Nanoparticles Intended for Drug Delivery. 2018, Springer. p. 103-124.
20. Wang, Y., et al., Multifunctional chitosan dopamine diatom-biosilica composite beads for rapid blood coagulation. Carbohydrate polymers, 2018. 200: p. 6-14.
21. Rao, S. B. and C. P. Sharma, Use of chitosan as a biomaterial: studies on its safety and hemostatic potential. Journal of Biomedical Materials Research: An Official Journal of The Society for Biomaterials and The Japanese Society for Biomaterials, 1997. 34(1): p. 21-28.
22. Gordy, S. D., P. Rhee, and M. A. Schreiber, Military applications of novel hemostatic devices. Expert review of medical devices, 2011. 8(1): p. 41-47.
23. O'Shaughnessy, D. and R. Gill, 19 Cardiothoracic surgery. Practical Hemostasis and Thrombosis, 2009: p. 194.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, products specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

While the present disclosure has been disclosed with references to certain embodiments, numerous modifications, alterations, and changes to the described embodiments are possible without departing from the sphere and scope of the present disclosure, as defined in the appended claims. Accordingly, it is intended that the present disclosure is not limited to the described embodiments, but that it has the full scope defined by the language of the following claims, and equivalents thereof.

1. A method of stopping bleeding comprising:
applying a peptide-based hemostatic material to a bleeding wound,
wherein the peptide-based hemostatic material comprises at least one self-assembling ultrashort peptide, and
wherein an application of the peptide-based hemostatic material to the bleeding wound produces a coagulation time of less than 40 seconds.

2. The method of claim 1, wherein the at least one self-assembling ultrashort peptide has a general formula selected from: 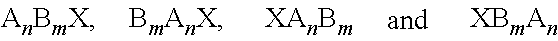
wherein the total number of amino acids of the ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
wherein X is comprised of a polar amino acid, selected from the group consisting of: aspartic acid, glutamic acid, lysine, arginine, histidine, cysteine, serine, threonine, asparagine, and glutamine.

3. The method of claim 1, wherein the self-assembling ultrashort peptide comprises at least one selected from the group consisting of IVFK (SEQ ID NO. 1), IVZK (SEQ ID NO. 2), KFVI (SEQ ID No. 3) and KZVI (SEQ ID No. 4),
wherein I is isoleucine, F is phenylalanine, K is lysine, V is valine and Z is cyclohexylalanine.

4. The method of claim 1, wherein the at least one self-assembling ultrashort peptide further comprises an N-terminal protecting group and a C-terminal protecting group.

5. The method of claim 4, wherein the N-terminal protecting group is an acetylated group and the C-terminal protecting group is an amidated group.

6. The method of claim 1, wherein the concentration of the self-assembling ultrashort self-assembling peptide is at least 3 mM.

7. The method of claim 1, wherein the coagulation is independent of fibrin.

8. The method of claim 1, wherein the coagulation is independent of coagulation factors.

9. The method of claim 1, wherein the at least one self-assembling ultrashort peptide is not toxic to cells.

10. A method of stopping bleeding comprising:
applying a peptide-based hemostatic material to a bleeding wound,
wherein the peptide-based hemostatic material comprises at least one self-assembling ultrashort peptide,
wherein an application of the peptide-based hemostatic material to the bleeding wound produces a coagulation time of less than 40 seconds,
wherein the self-assembling ultrashort peptide has a general formula selected from: 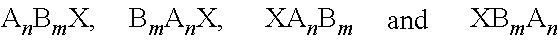
wherein the total number of amino acids of the self-assembling ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
wherein X is comprised of a polar amino acid, selected from the group consisting of: aspartic acid, glutamic acid, lysine, arginine, histidine, cysteine, serine, threonine, asparagine, and glutamine.

11. The method of stopping bleeding in claim 10, wherein the peptide-based hemostatic material is applied using a hand-held hemostatic device comprising:
a body of the hand-held device;
at least one bioink cartridge;
at least one pumping mechanism;
at least one control button;
at least one power system; and
at least one nozzle,
wherein the at least one pump is connected to the at least one bioink cartridge through at least one tubing, and
wherein the at least one nozzle is located at one end of the body of the hand-held device.

12. The method of stopping bleeding of claim 11, wherein the at least one power system is at least one selected from the group consisting of solar power panel and battery.

13. The method of stopping bleeding of claim 10, wherein the pumping mechanism is screw-driven piston.

14. The method of stopping bleeding of claim 10, wherein the hand-held hemostatic device further comprises a screen on the surface of the body of the hand-held device, wherein the screen displays status and parameters related to the devices' operation.

15. The method of stopping bleeding of claim 10, wherein the hand-held hemostatic device further comprises at least one heating or cooling mechanism.

16. The method of stopping bleeding of claim 10, wherein the at least one nozzle is interchangeable.

17. The method of stopping bleeding of claim 10, wherein the hand-held hemostatic device is water-proofing.

18. The method of stopping bleeding of claim 10, wherein the hand-held hemostatic device further comprises at least one camera at the same end of the body of the hand-held device as the nozzle, wherein the view of the camera can be streamed to a remote device.

19. The method of stopping bleeding of claim 18, wherein the remote device is a smartphone.

20. The method of stopping bleeding of claim 10, wherein the at least one bioink cartridge comprises at least 2 compartments containing the self-assembling ultrashort peptide and activation fluid in separate compartments.

21. The method of stopping bleeding of claim 10, wherein the flow rate of the peptide-based hemostatic material is controllable using the at least one control button.

22. An hand-held hemostatic device comprising:
a body of the hand-held device;
at least one bioink cartridge;
at least one pumping mechanism;
at least one control button;
at least one power system; and
at least one nozzle,
wherein the at least one pump is connected to the at least one bioink cartridge through at least one tubing,
wherein the at least one nozzle is located at one end of the body of the hand-held device; and
wherein the hand-held hemostatic device contains a peptide-based hemostatic material comprising at least one self-assembling ultrashort peptide which has a coagulation time of less than 40 seconds.

23. The hand-held hemostatic device of claim 22, wherein the at least one self-assembling ultrashort peptide has a general formula selected from: 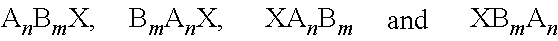
wherein the total number of amino acids of the ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
wherein X is comprised of a polar amino acid, selected from the group consisting of: aspartic acid, glutamic acid, lysine, arginine, histidine, cysteine, serine, threonine, asparagine, and glutamine.

24. The hand-held hemostatic device of claim 22, wherein the self-assembling ultrashort peptide comprises at least one selected from the group consisting of IVFK (SEQ ID NO. 1), IVZK (SEQ ID NO. 2), KFVI (SEQ ID No. 3) and KZVI (SEQ ID No. 4),
wherein I is isoleucine, F is phenylalanine, K is lysine, V is valine and Z is cyclohexylalanine.

25. The hand-held hemostatic device of claim 22, wherein the at least one self-assembling ultrashort peptide further comprises an N-terminal protecting group and a C-terminal protecting group.

26. The hand-held hemostatic device of claim 25, wherein the N-terminal protecting group is an acetylated group and the C-terminal protecting group is an amidated group.

27. The hand-held hemostatic device of claim 22, wherein the concentration of the self-assembling ultrashort self-assembling peptide is at least 3 mM.

28. The hand-held hemostatic device of claim 22, wherein the coagulation is independent of fibrin.

29. The hand-held hemostatic device of claim 22, wherein the coagulation is independent of coagulation factors.

30. The hand-held hemostatic device of claim 22, wherein the at least one self-assembling ultrashort peptide is not toxic to cells.
